(19) United States
(12) Patent Application Publication
(10) Pub. No.: US 2020/0383600 A1
(43) Pub. Date: Dec. 10, 2020

(54) ANALYTE SENSOR

(71) Applicant: PercuSense, Inc., Valencia, CA (US)

(72) Inventors: RAJIV SHAH, RANCHO PALOS VERDES, CA (US); KATHERINE WOLFE, DUNWOODY, GA (US); SHAUN PENDO, WOFFORD HEIGHTS, CA (US)

(73) Assignee: PercuSense, Inc., Valencia, CA (US)

(21) Appl. No.: 16/894,822

(22) Filed: Jun. 7, 2020

Related U.S. Application Data
(60) Provisional application No. 62/858,993, filed on Jun. 8, 2019.

(51) Int. Cl. A61B 5/053; A61B 5/145

(52) U.S. Cl. CPC A61B 5/053; A61B 5/14532

(57) ABSTRACT

A sensor assembly is disclosed. The sensor assembly having a first conductor and a second conductor being separated by an insulator. The sensor assembly further has an aperture that is formed through the first conductor, the second conductor and the insulator, wherein formation of the aperture creates an electrical short circuit between the first conductor and the second conductor.

### RELATED APPLICATIONS

This application claims the benefit of U.S. provisional application No. 62/858,993 filed Jun. 8, 2019. The application listed above is hereby incorporated by reference in its entirety for all purposes.

### FIELD OF THE INVENTION

The present invention is generally directed to devices that perform in vivo monitoring of at least one physiological parameter such as, but not limited to, perfusion, temperature or concentration of at least one analyte. In particular, the present invention is directed toward minimally invasive sensors that provide real-time information regarding the presence or concentration of an analyte or analytes such as, but not limited to, glucose, oxygen or lactate within a subject.

### BACKGROUND OF THE INVENTION

Diabetes is a growing healthcare crisis, affecting nearly 30 million people in the United States. Approximately 10 percent of those affected require intensive glucose and insulin management. In hospital patients, hypoglycemia in both diabetic and non-diabetic patients is associated with increased cost and short- and long-term mortality.

To prevent complications, diabetes requires ongoing management. Continuous glucose monitoring (CGM) has been shown in studies to be the most effective way to improve glucose control, whether used with insulin injections or a continuous insulin pump. CGM systems typically rely on sensors that are implanted under the skin for time periods varying between days and weeks. Efficacy of CGM can be further enhanced by monitoring additional analytes such as, but not limited to lactate and/or ketones. These continuous multianalyte sensors can improve insight into metabolic status that can lead to more personalized therapy or treatment that improves both short and long term patient outcomes.

However, improvements in care and outcome would ideally not come at the expense of user comfort and convenience. Having to insert multiple single analyte sensors to achieve multianalyte capability or having to insert a single relatively large multianalyte sensor may be perceived as encumbrances that dissuade users from adopting a continuous multianalyte sensor system. Thus, it continues to be advantageous to minimize the physical size of any implanted device.

Accordingly, it would be highly advantageous to enable the ability to selectively electrically short separate conductors within a single sensor assembly. The claimed invention seeks to address many issues associated with selectively electrically shorting separate conductors.

### BRIEF SUMMARY OF THE INVENTION

In one embodiment, a sensor assembly is disclosed. The sensor assembly having a first conductor and a second conductor being separated by an insulator. The sensor assembly further has an aperture that is formed through the first conductor, the second conductor and the insulator, wherein formation of the aperture creates an electrical short circuit between the first conductor and the second conductor.

In another embodiment a method of forming a sensor is disclosed. The method includes the operations of forming a first conductor having a first contact pad and forming a second conductor having a second contact pad. The method further includes the operations of separating the first conductor from the second conductor with an insulator and creating an aperture through the first conductor, the second conductor and the insulator, the creation of the aperture electrically shorting the first conductor to the second conductor.

In still another embodiment, a method of forming a sensor is disclosed. The method includes the operations to form a sensor with a first side and a second side, the second side being opposite the first side. The method further includes an operation to form a first conductor with a first contact pad on the first side and an operation to form a second conductor with a second contact pad on the second side. An additional operation separates the first conductor from the second conductor with an insulator. Further operations create a first aperture through the first conductor and the insulator and also create a second aperture through the second conductor and the insulator. Still additional operations fill the first aperture with a first supplemental conductor and further fill the second aperture with a second supplemental conductor. Wherein the first supplement conductor enables electrical contact to the first conductor on at least one of the first side or the second side and the second supplemental conductor enables electrical contact to at least one of the first side or the second side.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1 is a pseudo isometric exploded illustration of exemplary components within a sensor assembly, in accordance with embodiments of the present invention.

FIG. 2 is a pseudo isometric exploded view of a portion of the multilayer structure of the sensor, in accordance with embodiments of the present invention, in accordance with embodiments of the present invention.

FIG. 3A is a pseudo-isometric view of a portion of sensor with apertures, in accordance with embodiments of the present invention.

FIG. 3B is a pseudo-isometric view of an electrical short between the first conductor and the second conductor that is formed during the creation of the aperture, in accordance with embodiments of the present invention.

FIGS. 4A-4C are exemplary pseudo-isometric views of alternative embodiments of the sensor assembly, in accordance with embodiments of the present invention.

FIG. 5 is an exemplary cross-section of a variation of the multilayer structure in FIG. 4C that illustrates how apertures that do not electrically short the first and second conductors can enable electrical contact with the conductors from different or same sides of the sensor, in accordance with embodiments of the present invention.

FIG. 6 is a flow chart illustrating exemplary operations to form a sensor with a selective electrical short circuit, in accordance with embodiments of the present invention.

FIG. 7 is a flow chart illustrating exemplary operations to form a sensor with apertures that enable electrical connections to be made to electrically isolated conductors from various sides of a sensor, in accordance with embodiments of the present invention.

### DETAILED DESCRIPTION

Presented below are embodiments that are intended to enable selectively creating an electrical short circuit between electrically isolated conductors. The ability to selectively create an electrical short circuit between electrically isolated conductors can enable minimally invasive implantable sensors to be miniaturized resulting in reduced insertion force and physical discomfort. In some embodiments, particularly those having the ability to measure multiple analytes using multiple conductors, each conductor can have their own respective working electrode, counter electrode and reference electrode. In alternative embodiments, each conductor can have their own respective working electrode and a combined counter/reference electrode, or pseudo-reference electrode. In still further alternative embodiments, the individual conductors can each have their own working electrode while sharing a common counter/reference (pseudo-reference) electrode.

The ability to selectively create the electrical short circuit between the conductors enables the shared counter/reference electrode. Thus, the selective electrical short circuit enables sensors that can be physically smaller because a single counter/reference electrode replaces two counter/reference electrodes or alternatively, two counter electrodes and two reference electrodes. From another perspective, the selective electrical short circuit enables increased area for a working electrode because the single counter/reference electrode has emancipated area on one of the conductors that would have been dedicated to a counter electrode and a reference electrode, or a counter/reference electrode. Though various features of different embodiments may be discussed individually, the various features and embodiments should be viewed as potentially being combined within another embodiment or other embodiments so long the intended operation of the combined embodiments is not compromised. Accordingly, the features described in each embodiment should be viewed as being combinable with the other features and embodiments discussed within the following pages.

In many examples discussed below sensor structures are discussed in reference to a sensor having two conductors. While embodiments and examples may be related to figures having a specific number of conductors capable of detecting a specific analyte or analytes, the scope of the disclosure and claims should not be construed to be limited to the number of conductors or analytes illustrated or discussed below. Rather it should be recognized that fewer or even additional conductors can assist or enable in the detection, diagnosis and monitoring of various metabolic conditions or general physiological health.

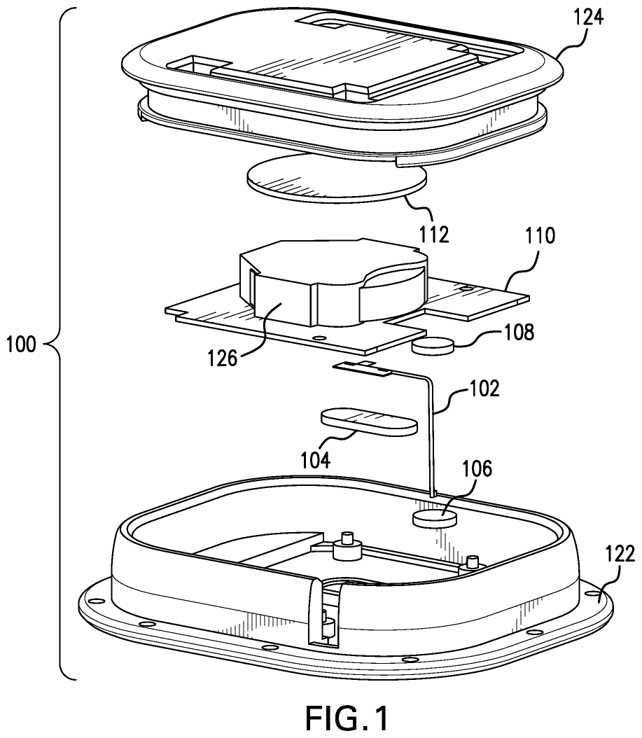

FIG. 1 is a pseudo isometric exploded illustration of exemplary components within a sensor assembly 100, in accordance with embodiments of the present invention. The individual components within the sensor assembly 100 are intended to be exemplary of various components that may be found within a sensor assembly and should not be construed as limiting. Additional embodiments of sensor assemblies 100 may include fewer or additional components. Sensor assembly 100 includes a sensor 102. Sensor 102 may have various configurations, while preferred embodiments of sensor 102 include attributes and characteristics such as those discussed in U.S. patent application Ser. No. 15/472,194 filed on Mar. 28, 2017 and Ser. No. 16/152,727 filed on Oct. 5, 2018; along with PCT serial number PCT/US2018/038984 filed on Jun. 22, 2018, which are hereby incorporated by reference for all purposes. In many embodiments, the sensor 102 is configured to measure real-time concentrations of at least one or more analytes in vivo such as, but not limited to glucose, lactate, ketones, oxygen, reactive oxygen species and the like. In some embodiments the sensor 102 acquires in vivo measurement of an analyte while placed within at least one or more locations such as, but not limited to subcutaneous tissue, muscle tissue, intravenously or the like.

The sensor assembly 100 further includes a circuit board 110. The circuit board 110 may be comprised of multiple layers or rigid material, flexible material, or combinations thereof. The circuit board 110 enables electrical connection between the sensor 102 various electronic components such as, but not limited to a power supply 126, memory (not shown), radios to enable bidirectional communication (not shown) and an application specific integrated circuit (ASIC) (not shown). In some embodiments the electrical connection between the circuit board 110 and the sensor 102 is achieved via compression of conductive elastomers placed between the sensor 102 and the circuit board 110. In other embodiments, protrusions such as pins or bumps extend from the circuit board 110 to make electrical contact with contact pads on the sensor 102.

Conformal material 104 and 112 are selectively positioned within the sensor assembly 100 between the case top 124 and a case bottom. In one embodiment, the conformal material 104 is positioned between the case bottom 122 and the sensor 102. The durometer of conformal material 104 is selected to obtain sufficient compression to ensure electrical contact between the sensor 102 and the circuit board 110. Similarly, conformal material 112 is positioned between the case top 124 and the circuit board 110. The durometer of conformal material 112 also being selected to provide sufficient compression to ensure electrical contact between the sensor 102 and the circuit board.

The case bottom 122 includes an opening that enables a distal end of the sensor 102 to extend through the case bottom 122. The sensor passes through a bottom seal 106. The bottom seal 106 is selected from a flexible material that creates a fluid barrier to prevent fluids such as liquids from being introduced within an interior of the sensor assembly 100. In some embodiments the bottom seal 106 is molded into the case bottom 122. In other embodiments the bottom seal 106 is a discrete component that is placed within an opening in the case bottom 122. While a single bottom seal 106 is shown in FIG. 1, other embodiments can include additional seals that prevent fluid intrusion within the case bottom. However, should fluid pass through the bottom seal 106, the assembly further includes a top seal 108. The top seal 108 is intended to prevent ingressed fluid from making contact with the circuit board 110. The specific elements shown in FIG. 1 and discussed above are intended to be illustrative and should not be construed as limiting.

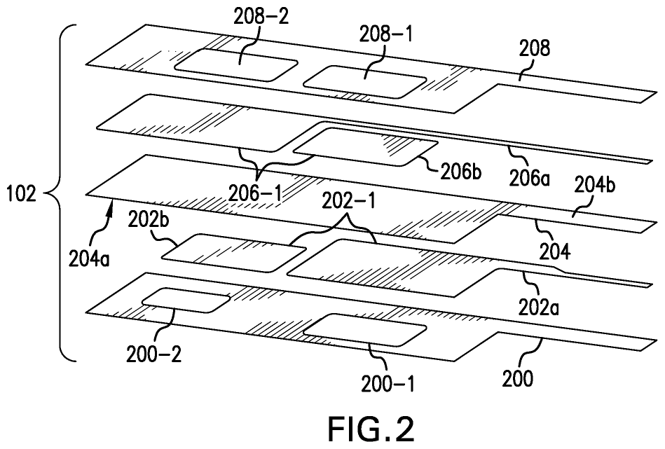

FIG. 2 is a pseudo isometric exploded view of a portion of the multilayer structure of the sensor 102, in accordance with embodiments of the present invention. The portion of the sensor 102 illustrated in FIG. 2 is intended to illustrate one embodiment of electrical contact pads for the sensor 102. In this embodiment the sensor 102 includes a first conductor 202-1 and a second conductor 206-1. The first conductor 202-1 can be further broken down into a first portion 202a and a second portion 202b. Likewise, the second conductor 206-1 can be viewed as having a first area 206a and second area 206b. In many embodiments the first conductor 202-1 and the second conductor 206-1 are conductive alloys having a thickness between approximately 0.00005 and 0.01 inches. In particular embodiments, the first and second conductors 202-1 and 206-1 are selected from stainless steel alloys having a thickness between approximately 0.0005 and 0.003 inches. In some embodiments, the first conductor 202-1 and the second conductor 206-1 are selected to have identical material properties (e.g. elasticity, ductility, fatigue limit plasticity and the like) and thicknesses. In other embodiments, the first conductor 202-1 and the second conductor 206-1 have different material properties while having identical or substantially identical thicknesses. In still other embodiments, the first conductor 202-1 and the second conductor 206-1 are selected to have different material properties and different thicknesses.

Separating the first conductor 202-1 and the second conductor 206-1 is a central insulator 204. Typically, the central insulator 204 is coupled to both the first conductor 202-1 and the second conductor 206-1 using an adhesive. In many embodiments the central insulator 204 is a material such as, but not limited to polyimide film. The sensor 102 further includes bottom insulation 200 that contains bottom opening 200-1 and optional bottom opening 200-2. The bottom opening 200-1 and optional bottom opening 200-2 define contact pads on the first portion 202a and optional second portion 202b of the first conductor. Similarly, top insulation 208 includes top opening 208-1 and optional top opening 208-2. Top opening 208-1 defines a contact pad for the second area 206b, while the top opening 208-2 defines a contact pad on the first area 206a of the second conductor 206-1.

In many embodiments the bottom insulation 200 and the top insulation 208 are the same material as the central insulator 204. Also, like the central insulator 204, the bottom insulation 200 and the top insulation 208 may be made from polyimide film and coupled to their respective conductors via an adhesive. The specific embodiments discussed above should not be construed as limiting. Various embodiments of the sensor 102 can include additional or different insulators and/or insulation materials, incorporate different or additional coupling mechanisms or different or additional materials for the first conductor and/or the second conductor. Furthermore, while the embodiments illustrated in FIG. 2 includes two conductors, other embodiments can include additional conductors that are separated by additional insulators.

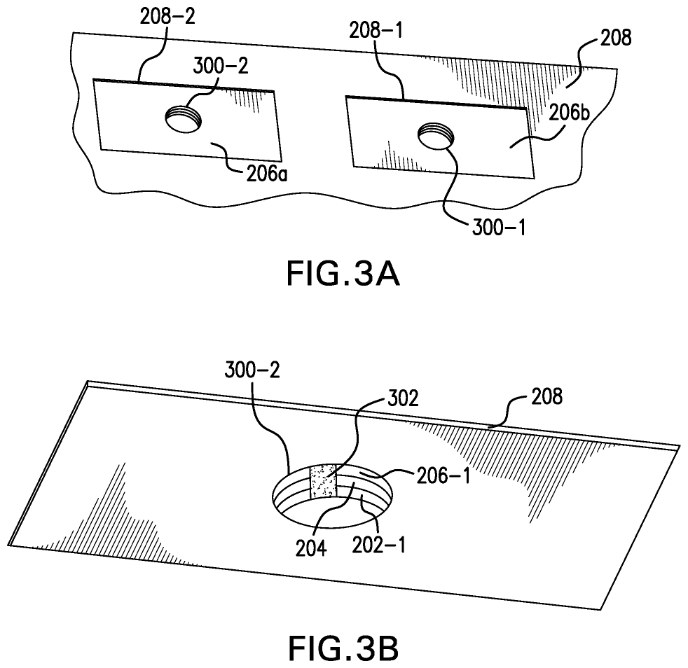

FIG. 3A is a pseudo-isometric view of a portion of sensor 102 with apertures 300-1 and 300-2, in accordance with embodiments of the present invention. Apertures 300-1 and 300-2 are formed within an area exposed by top openings 208-1 and 208-2 within the top insulation 208. The relative size and shape of apertures 300-1 and 300-2 along with top openings 208-1 and 208-2 are intended to be illustrative rather than limiting. For example, while the illustrations in FIG. 3A have the top openings 208-1 and 208-2 being substantially the same size and shape, in alternative embodiments the top openings 208-1 and 208-2 can be different sizes and different shapes. Similarly the apertures 300-1 and 300-2 may be different sizes and shapes as well. While FIG. 3A includes two apertures, aperture 300-1 and and aperture 300-2, in many embodiments a single aperture is formed within either top opening 208-1 or 208-2. Alternatively, in other embodiments, more than two apertures can be formed.

In many embodiments the apertures 300-1 and 300-2 are formed using a laser. Alternative embodiments form the apertures 300-1 and 300-2 using techniques such as, but not limited to punches or drills. In embodiments having more than one aperture, the apertures can be formed using the same technique while in other embodiments different techniques are used to form the multiple apertures. The formation of the aperture is intended to create a permanent electrical short between the first conductor 202-1 and the second conductor 206-1.

FIG. 3B is a pseudo-isometric view of an aperture 300-2 that further includes an electrical short created by slag 302 between the first conductor 202-1 and the second conductor 206-1, in accordance with embodiments of the present invention. In embodiments where the aperture 300-2 is formed using a laser, slag created from either or both the first conductor 202-1 or the second conductor 206-1 creates an electrical short circuit between the first conductor 202-1 and the second conductor 206-1. The creation of slag is dependent on the energy and duration of the laser pulse along with the mechanical properties such as, but not limited to, thickness of the first conductor 202-1, thickness of the second conductor 206-1. Beam focus of the laser, direction of travel of the laser and/or relative motion to the sensor to the laser, and angle of incidence of the laser beam to the sensor, or various combinations thereof can be optimized or tuned to repeatedly and robustly create an electrical short circuit with laser generated slag.

In embodiments where alternative mechanical techniques such as, but not limited to, drilling or punching are used to create the aperture 300-2, speed and sharpness of the drills or punches can be optimized to enable creation of an electrical short circuit between the first conductor 202-1 and the second conductor 206-1. The techniques described above are intended to be illustrative on preferred embodiments to create an electrical short circuit between the first conductor and the second conductor. Various other mechanical, electro-optical or other techniques may be used so long as the electrical short circuit between the first conductor and the second conductor is established as part of, or during the creation of the aperture. Furthermore, while the apertures 300-1 and 300-2 are illustrated as being through holes passing through the entirety of the sensor structure, in other embodiments an aperture may be a blind hole. In embodiments having multiple apertures, some apertures may be blind holes while other apertures are through holes.

In the embodiment illustrated in FIG. 3B an electrical short circuit is created between the first and second conductors by slag 302 generated during the forming of the aperture 300-2. In alternative embodiments, an electrical short circuit between the first and second conductors can be formed not within an aperture 300-2, but along an edge 304 (FIG. 3A). Creation of an electrical short along the edge 304 can be accomplished during a singulation procedure where individual sensors are separated from a substrate via a laser cutting process. In some embodiments a laser cutting process can include various settings for movement dwell times and laser power. A first setting can be used to cleanly cut (singulate) the sensor while a second setting can be used to create slag to enable the electrical short circuit. In some embodiments, the electrical short circuit is formed first followed by singulation. In other embodiments, singulation is initiated and creation of the electrical short completely separates an individual sensor from the substrate.

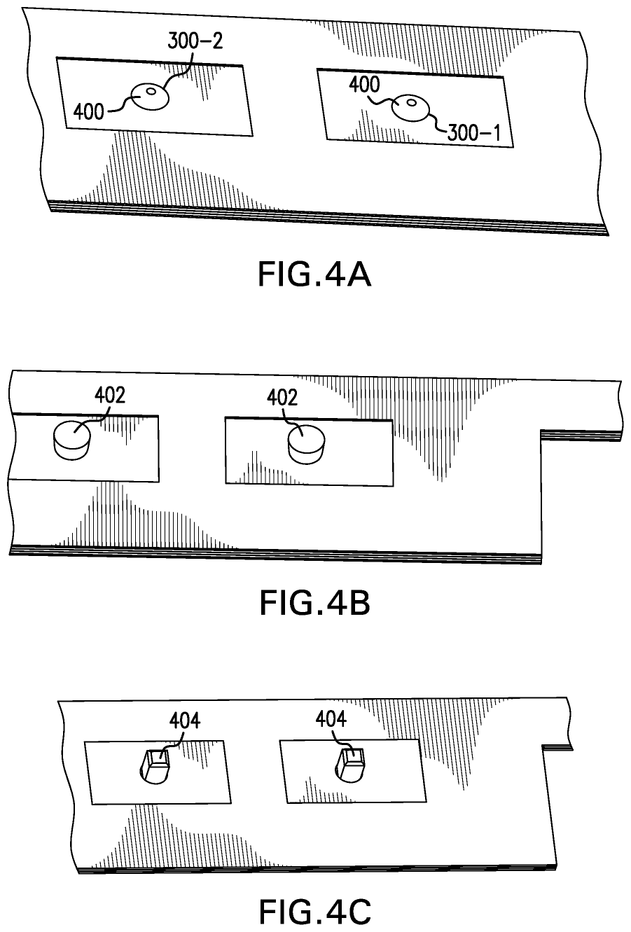

FIGS. 4A-4C are exemplary pseudo-isometric views of alternate embodiments of the sensor assembly 102 that include a supplemental short circuit, in accordance with embodiments of the present invention. In some embodiments it may be desirable to supplementally short circuit the first and second conductor. In many embodiments, the use of a supplemental short circuit can also provide additional benefits of mechanical reinforcement of the electrical short circuit between the first and second conductors or enabling the ability to physically locate the sensor relative to the circuit board.

FIG. 4A is an exemplary illustration of apertures 300-1 and 300-2 being filled with material 400. In many embodiments the material 400 is selected from materials that are electrically conductive, thereby providing additional or supplemental short circuit capability beyond the electrical short circuit created during formation of the aperture. Exemplary material 400 include, but are not limited to, conductive epoxies and the like.

In some embodiments the aperture can be overfilled or underfilled with the material 400 resulting in the material 400 extending beyond or below the surface of the first or second conductor exposed within the top opening or bottom opening. In other embodiments, the aperture can be filled such that the material 400 is substantially coincident with the first or second conductor exposed within the top opening or bottom opening. Underfill, overfill or coincident application of the material 400 within the aperture may be selected based on requirements for the electrical contact pads onto which they are disposed.

FIG. 4B is an exemplary illustration of plugs 402 being inserted into the apertures 300-1 and 300-2. The plugs 400 can be selected from conductive materials such as, but not limited to conductive elastomers and the like to enable a supplemental electrical short circuit between the first conductor and the second conductor within the aperture. FIG. 4C is an exemplary illustration of pins 404 within the apertures 300-1 and 300-2. In many embodiments the pins 404 include conductive materials such as, but not limited to copper, silver, gold and the like. In some embodiments the pins 404 are coupled to the circuit board (FIG. 1) and in addition to supplementally electrically short circuiting the first and second conductors, the pins 404 physically locate the sensor 102 relative to the circuit board. Additionally, the pins can provide electrical connectivity to the circuit board along with supplementing the electrical short circuit between the first conductor and the second conductor.

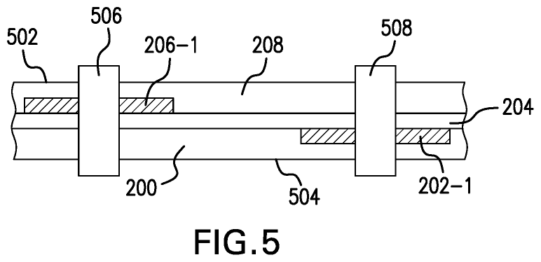

FIG. 5 is an exemplary cross-section of a variation of the multilayer structure in FIG. 4C that illustrates how apertures that do not electrically short the first and second conductors can enable electrical contact with the conductors from different or same sides of the sensor, in accordance with embodiments of the present invention. As illustrated in FIG. 5, the first conductor 202-1 and the second conductor 206-1 are separated by the central insulator 204. In this particular embodiment, the first conductor 202-1 and the second conductor 206-1 are not electrically short circuited during the creation of an aperture that traverses the top insulation 208 the second conductor, the central insulator 204 and the bottom insulation 200 as well as an aperture that traverses the bottom insulation 200, the first conductor 202-1, the central insulator 204 and the top insulation 208. Supplemental conductor 506 and second supplemental conductor 508 are inserted within the respective apertures and enable electrical contact to be made with either or both of the respective conductors from a first side 502 or a second side 504.

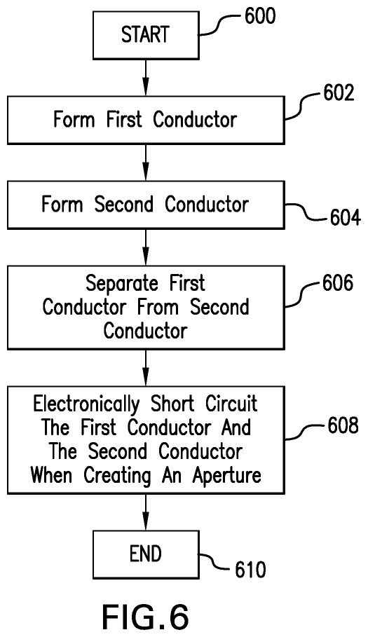

FIG. 6 is a flow chart illustrating exemplary operations to form a sensor with a selective electrical short circuit, in accordance with embodiments of the present invention. The operations begin with a start operation 600. Operation 602 forms a first conductor while operation 604 forms a second conductor. In many embodiments the first conductor includes a first contact pad and the second conductor includes a second contact pad. In other embodiments the first and second contact pads are created in a subsequent or separate operation. Operation 606 separates the first conductor and the second conductor with an insulator. In various embodiments, portions of this operation may be optional as the first or second conductor may include an insulator that is coupled with the other conductor.

Operation 608 creates an aperture. The creation of the aperture in operation 608 also creates an electrical short circuit between the first conductor and the second conductor. In many embodiments operation 608 is accomplished with a laser. The laser being configured to generate slag that creates the electrical short circuit between the conductors. In other embodiments operation 608 is accomplished with a drill. Drill parameters such as, but not limited to rotational speed of the drill bit along with translation speed of the drill bit through the sensor can be configured or tuned to achieve the desired short circuit between the first and second conductors. In still other embodiments operation 608 is accomplished with a punch. As with the drill, punch parameters may require tuning in order to achieve the desired electrical short circuit between the first and second conductors. Operation 610 ends the process and completes the flowchart.

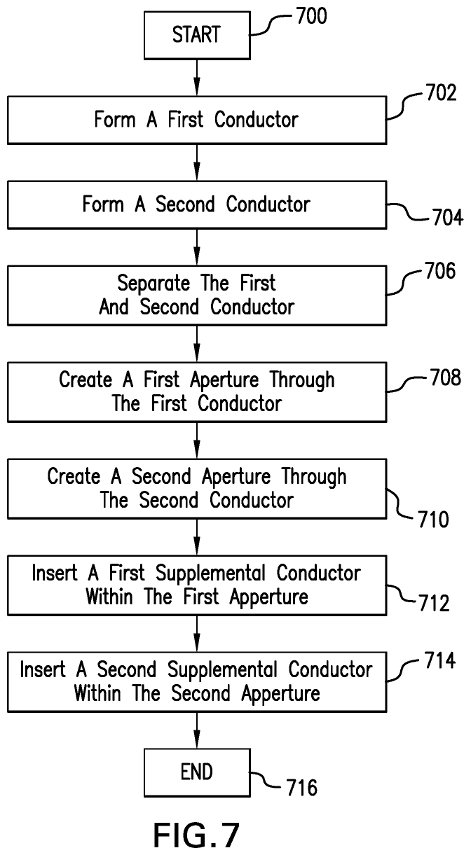

FIG. 7 is a flow chart illustrating exemplary operations to form a sensor with apertures that enable electrical connections to be made to electrically isolated conductors from various sides of a sensor, in accordance with embodiments of the present invention. The operations begin with start operation 700. Operation 700 forms a first conductor and operation 710 formed a second conductor. Operation 720 separates the first and second conductors using an insulator. Operation 708 creates a first aperture through the first conductor and the insulator. Operation 710 creates a second aperture through the second conductor and the insulator. Operation 712 results in a first supplemental conductor being inserted within the first aperture. Operation 714 results in a second supplemental conductor being inserted within the second aperture. The operations are concluded with end operation 716. Upon completion of the operations outlined in FIG. 7, electrical contact can be made to either the first conductor or the second conductor from either side of the sensor.

The specific operations, and particularly the order of operations, disclosed in the embodiments associated with FIGS. 6 and 7 are intended to be exemplary and should not be construed as limiting. In various other embodiments the operations discussed can be performed in a multitude of different orders. In still other embodiments an operation discussed above can be divided into separate operations. Similarly, multiple operations discussed above can be combined into a single operation.

The embodiments discussed above are intended to be exemplary. For example, while many of the embodiments are related to sensing using two conductors, other embodiments can be related to generic subdermal sensing using a single conductor or a plurality of conductors to enable sensing or detection of analytes or compounds such as, but not limited to lactate, ketones, oxygen, glucose, reactive oxygen species and the like. Additionally, while many of the embodiments shown in the accompanying figures include a single aperture, various other embodiments can include multiple apertures, where creation of each aperture results in an electrical short circuit. Furthermore, the circular apertures shown in the accompanying figures should not be construed as limiting. Apertures can be formed in various shapes, sizes and at angles other than perpendicular to the sensor such as oblique or acute angles.

In many embodiments, additional features or elements can be included or added to the exemplary features described above. Alternatively, in other embodiments, fewer features or elements can be included or removed from the exemplary features described above. In still other embodiments, where possible, combination of elements or features discussed or disclosed incongruously may be combined together in a single embodiment rather than discreetly as in the exemplary discussion.

Accordingly, while the description above refers to particular embodiments of the invention, it will be understood that many modifications may be made without departing from the spirit thereof. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

1. A sensor, comprising:
a first conductor and a second conductor being separated by an insulator, and
an aperture being formed through the first conductor, the second conductor and the insulator, wherein formation of the aperture creates an electrical short circuit between the first conductor and the second conductor.

2. The sensor of claim 1, wherein the insulator is a central insulator having a first side and second side, the first conductor being coupled to the first side and the second conductor being coupled to the second side.

3. The sensor of claim 2, wherein the first conductor includes a first portion and a second portion.

4. The sensor of claim 3, wherein the second conductor includes a first area and a second area.

5. The sensor of claim 4, further comprising:
a bottom insulator being coupled to the first conductor, the bottom insulator having at least one bottom opening.

6. The sensor of claim 5, further comprising:
a top insulator being coupled to the second conductor, the top insulator having at least one top opening.

7. The sensor of claim 6, wherein the bottom opening defines a bottom contact pad and the top opening defines a top contact pad.

8. The sensor of claim 7, wherein the aperture is formed between the bottom contact pad and the top contact pad.

9. The sensor of claim 2, wherein the aperture is formed via a laser, the laser being configured to generate and deposit slag between the first conductor, the second conductor and the insulator.

10. The sensor of claim 9, further comprising:
a supplemental short circuit within the aperture, the supplemental short circuit being an electrically conductive material.

11. A method of forming a sensor, the method comprising the operations of:
forming a first conductor having a first contact pad;
forming a second conductor having a second contact pad;
separating the first conductor from the second conductor with an insulator;
creating an aperture through the first conductor, the second conductor and the insulator, the creation of the aperture to electrically short circuit the first conductor and the second conductor.

12. The method of forming a sensor of claim 11, wherein the creation of the aperture is performed with a laser.

13. The method of forming a sensor of claim 11, further comprising the operation of:
supplementing the electrical short between the first conductor and the second conductor by filling the aperture with a conductive material.

14. The method of forming a sensor of claim 13, wherein the supplemental electrical short further enables relative location of the sensor within a sensor assembly.

15. A method of forming a sensor having a first side and a second side, the second side being opposite the first side, the method comprising the operations of:
forming a first conductor having a first area on the first side;
forming a second conductor having a second area on the second side;
separating the first conductor from the second conductor with an insulator
creating a first aperture through the first conductor and the insulator;
creating a second aperture through the second conductor and the insulator;
filling the first aperture with a first supplemental conductor; and
filling the second aperture with a second supplemental conductor,
wherein the first supplement conductor enables electrical contact to the first conductor on at least one of the first side or the second side and the second supplemental conductor enables electrical contact to at least one of the first side or the second side.

16. The method of forming a sensor of claim 15, wherein electrical contact to the first conductor is made on the first side and electrical contact to the second conductor is made on the first side.

17. The method of forming a sensor in claim 15, wherein electrical contact to the first conductor is made on the second side and electrical contact to the second conductor is made on the first side.

18. The method of forming a sensor in claim 15, wherein electrical contact to the first conductor is made on the second side and electrical contact to the second conductor is made on the second side.

19. The method of forming a sensor in claim 15, wherein electrical contact to the first conductor is made on the first side and electrical contact to the second conductor is made on the second side.

20. The method of forming a sensor in claim 15, electrical contact to the first conductor and electrical contact to the second conductor is made on either the first side or the second side.
